(19) United States
(12) Patent Application Publication
(10) Pub. No.: US 2026/0242442 A1
(43) Pub. Date: Aug. 20, 2026

(54) CHIMERIC CD8-ALPHA CO-RECEPTOR COMPOSITIONS AND METHODS OF USE

(71) Applicant: Poseida Therapeutics, Inc., San Diego, CA (US)

(72) Inventors: Lu YAO, San Diego, CA (US); Sergio QUINONES-PARRA, San Diego, CA (US); Julia CORONELLA, Carlsbad, CA (US); Devon SHEDLOCK, San Diego, CA (US)

(21) Appl. No.: 19/100,013

(22) PCT Filed: Aug. 10, 2023
(86) PCT No.: PCT/US2023/072022

§ 371 Date: Jan. 30, 2025

Related U.S. Application Data
(60) Provisional application No. 63/371,136, filed on Aug. 11, 2022.

(51) Int. Cl. C07K 14/705; A61K 35/17; A61K 40/11; A61K 40/42; A61P 35/00; C12N 5/0783

(52) U.S. Cl. CPC C07K 14/70517; A61K 35/17; A61K 40/11; A61K 40/4224; A61P 35/00; C07K 14/70514; C12N 5/0636; A61K 2239/15; A61K 2239/21; A61K 2239/22; A61K 2239/57; C07K 2319/01; C07K 2319/02; C07K 2319/03; C07K 2319/90; C12N 2510/00

(57) ABSTRACT

Disclosed are chimeric CD8α co-receptor compositions and methods of use therefor. In particular, disclosed are chimeric CD8α homodimer co-receptor compositions, cells comprising the chimeric co-receptors, and pharmaceutical compositions comprising the co-receptors and/or cells. Methods of making and using the co-receptor compositions also are provided.

### RELATED APPLICATIONS

This application claims priority to, and the benefit of, U.S. Provisional Application 63/371,136, filed Aug. 11, 2022, the entire contents of which are incorporated herein by reference.

### TECHNICAL FIELDS

The disclosure is directed to chimeric CD8α co-receptor compositions and methods of use therefor. In particular, disclosed are chimeric CD8α homodimer co-receptor compositions, cells comprising the chimeric CD8α co-receptors, pharmaceutical compositions comprising the chimeric CD8α co-receptors and/or cells and methods of making and use thereof.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “POTH-074_001WO_SeqList_ST26.xml”. The XML file is 64,097 bytes, created on Jul. 24, 2023, and is being submitted electronically via USPTO Patent Center.

### BACKGROUND OF THE INVENTION

In recent years, adoptive cellular therapy using autologous T cells transduced to express T cells receptors (TCRs) or chimeric antigen receptor (CARs) has proven to be a very powerful approach for the treatment of diseases such as cancer. However, challenges remain, including uncoupling cytotoxicity against tumor cells from systemic toxicity. There has been a long felt but unmet medical need for compositions and methods for improving T-cell receptor (“TCR”)-mediated cytotoxicity of TCR-T cell therapies (“TCR-T therapies”). The disclosure provides a solution by providing a chimeric TCR, CD8α co-receptor that enhances the cytotoxic activity of TCR-T therapies.

### SUMMARY OF THE INVENTION

This disclosure provides a chimeric CD8α co-receptor, comprising: a) a truncated CD8α co-receptor, comprising a CD8α co-receptor extracellular domain and CD8α co-receptor transmembrane domain; and b) a CD4 intracellular domain comprising a palmitoylation motif and a Lck binding domain; wherein the CD4 intracellular domain is fused in frame to the C-terminus of the truncated CD8α co-receptor.

In some aspects, the truncated CD8 co-receptor comprises the amino acid sequence of SEQ ID NO: 1. In some aspects, the palmitoylation motif comprises the amino acid sequence of SEQ ID NO: 3. In some aspects, the Lck binding domain comprises the amino acid sequence of SEQ ID NO: 4. In some aspects, the CD4 intracellular domain comprising the palmitoylation motif and the Lck binding domain comprises the amino acid sequence of SEQ ID NO: 2.

In some aspects, the CD8α co-receptor comprises: a) a truncated CD8α co-receptor comprising the amino acid sequence of SEQ ID NO: 1; and b) a CD4 intracellular domain comprising a palmitoylation motif comprising the amino acid sequence of SEQ ID NO: 3 and a Lck binding domain comprising the amino acid sequence of SEQ ID NO: 4; wherein the CD4 intracellular domain is fused in frame to the C-terminus of the truncated CD8α co-receptor.

In some aspects, the CD8α co-receptor comprises: a) a truncated CD8α co-receptor comprising the amino acid sequence of SEQ ID NO: 1; and a CD4 intracellular domain comprising the amino acid sequence of SEQ ID NO: 2; wherein the CD4 intracellular domain is fused in frame to the C-terminus of the truncated CD8α co-receptor.

In some aspects, the chimeric CD8α co-receptor comprises the amino acid sequence of SEQ ID NO: 5.

This disclosure also provides a polynucleotide comprising a nucleic acid sequence encoding any one of the chimeric CD8α co-receptors of the disclosure. In some aspects, the polynucleotide is an mRNA molecule. In some aspects, the polynucleotide is a DNA molecule. In some aspects, the polynucleotide further comprises a promoter sequence operably linked with the DNA molecule to produce at least one mRNA molecule encoding the chimeric CD8α co-receptor in a cell.

This disclosure also provides a cell comprising the polynucleotide of the disclosure. In some aspects, the cell expresses the chimeric CD8α co-receptor. In some aspects, the cell further expresses a T-cell receptor (TCR). In some aspects, the cell further expresses a chimeric antigen receptor (CAR). In some aspects, the cell expresses a TCR and a CAR. In some aspects, the cell is a T-cell. In some aspects, the chimeric CD8α co-receptor is expressed as a homodimer on the cell membrane of the T-cell.

This disclosure also provides a pharmaceutical composition comprising any one of the cells of the disclosure and at least one pharmaceutically acceptable carrier or medicament.

This disclosure also provides a method of stimulating T cell Receptor (TCR)-mediated cytotoxicity of a population of T-cells in a subject in need thereof, comprising: a) introducing to a population of T cells, a polynucleotide encoding a TCR and a polynucleotide encoding any one of the chimeric CD8α co-receptor of the disclosure, wherein the plurality of T-cells in the population of T-cells co-expresses the TCR and the chimeric CD8α co-receptor on the cell membrane of the T-cell, and wherein the chimeric CD8α co-receptor is expressed as a homodimer; and b) administering the population of T-cells to the subject in need thereof, wherein the population of T-cells expressing the TCR and the chimeric CD8α co-receptor has a greater level of cytotoxicity in comparison to a population of T-cells expressing only the TCR. In some embodiments, the population of T-cells further expresses a CAR.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.100, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIGS. 1A-1B show schematics depicting an exemplary chimeric CD8α homodimer (chiCD8-homo-di) and CD8α heterodimer (CD8-hetero-di) and CD8α homodimer (CD8-homo-di) controls. FIG. 1A shows a chimeric CD8α homodimer co-receptor comprising a truncated CD8α co-receptor comprising only the CD8α co-receptor extracellular domain and the CD8α co-receptor transmembrane domain; a CD4 intracellular domain comprising a palmitoylation domain and a high-affinity Lck binding domain, wherein the CD4 intracellular domain is fused in frame to the C-terminus of the truncated CD8α co-receptor. FIG. 1B shows a control wildtype CD8α heterodimer co-receptor (CD8 hetero-di) and a CD8α homodimer co-receptor (CD8-homo-di).

FIG. 2 shows a schematic representation of a transposon designed for the dual expression of the chimeric CD8α co-receptor (“co-receptor”) and a TCR receptor comprised of a TCR alpha (“TCRa”) chain and TCR beta (“TCRb”) chain. A PGK promoter controls the expression of the chimeric CD8α co-receptor. A EF1a promoter controls the expression of the TCR receptor. ITR (inverted terminal repeat); Insul (insulator sequence); pA (polyA), iC9 (inducible proapoptotic polypeptide); DHFR (dihydrofolate reductase resistance cassette); TCRb (TCR beta chain); TCRa (TCR alpha chain).

FIGS. 3A-3B show a series of contour plots and graphs depicting flow cytometry sorting and in vitro cytotoxicity data for the chimeric CD8α co-receptor compared to TCR controls. FIG. 3A shows fluorescence activated cell sorting (FACS) analysis of TCR+CD8a+ (bottom row) and TCR+CD4+ (top row) T-cell populations co-expressing the chimeric CD8α co-receptor of the present disclosure (Chi_CD8 homo-di), a wild type CD8α co-receptor (CD8 Homo-di), a heterodimeric CD8α co-receptor (CD8 Hetero-di), or GFP compared to Mock controls. Percentage of cell population expressing receptors is as indicated in each panel. FIG. 3B shows two graphs depicting an in vitro cytotoxicity assay with a chimeric CD8α co-receptor compared to TCR controls in CD4+ (left) and CD8+ (right) T cells at the indicated ratios. The x-axis depicts time elapsed (in hours). The y-axis depicts tumor growth percentage (%), normalized to baseline. T cells are expressing co-receptors as indicated in the legend. This data shows superior in vitro cytotoxicity of the TCR+ chimeric CD8α co-receptor (TCR+chiCD8-homo-di) relative to all other controls (Mock, TCR+GFP, TCR+CD8-hetero-di, TCR+CD8-homo-di).

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### DETAILED DESCRIPTION OF THE INVENTION

The present invention relates to chimeric co-receptor compositions and methods of use therefor. In particular, the present invention relates to chimeric CD8α homodimer co-receptor compositions, cells comprising the chimeric CD8α co-receptors, pharmaceutical compositions comprising the CD8α co-receptors and/or cells and methods of use thereof.

This disclosure provides a chimeric CD8α co-receptor, comprising: a) a truncated CD8α co-receptor, comprising a CD8α co-receptor extracellular domain and CD8α co-receptor transmembrane domain; and b) a CD4 intracellular domain comprising a palmitoylation motif and a Lck binding domain; wherein the CD4 intracellular domain is fused in frame to the C-terminus of the truncated CD8α co-receptor.

The present disclosure overcomes problems associated with current technologies by providing chimeric CD8α co-receptor that can be expressed on the surface of T-cells and used in immunotherapies, such as for the treatment of immune-related diseases, including cancer and autoimmune disorders, as well as infection including but not limited to viruses. The present disclosure is based, at least in part, on the discovery that Palmitoylation motifs and Lck binding domains, which are absent from a wildtype CD8α co-receptor, allow recruitment of factors that improve T-cell signaling. An increase of T-cell signaling results in an increase of T-cell activation and cytotoxicity, which are advantageous traits for T cell therapeutics (such as TCR-T cells). Accordingly, the present disclosure provides cells and methods for generating cells (e.g., T cells) that express a chimeric CD8α co-receptor. Such cells exhibit increased T-cell activation and cytotoxicity against target cells.

### T-Cell Receptors

The T-cell receptor (“TCR”) is a molecule localized on the surface of T cells that is responsible for recognizing antigens bound to MHC molecules. During antigen processing, antigens are degraded inside cells and then carried to the cell surface in the form of peptides bound to major histocompatibility complex (MHC) molecules (human leukocyte antigen or HLA molecules in humans). T cells recognize these peptide-MHC complexes on the surface of antigen presenting cells or target tissue cells. Two classes of MHC molecules, MHC Class I and MHC Class II, deliver peptides from different cellular compartments to the cell surface where they are recognized by CD8+ and CD4+ T cells, respectively.

In particular, a TCR is a disulfide-linked membrane-anchored heterodimeric protein normally consisting of the highly variable alpha and beta chains that associate with the invariant CD3 chain molecules to form a complete functioning TCR. The alpha and beta chains are composed of extracellular domains comprising a Constant (C) region and a Variable (V) region. The Constant region is proximal to the cell membrane, followed by a transmembrane region and a short cytoplasmic tail, while the Variable region binds to the ligand. The variable domain of both the TCR alpha-chain and beta-chain each have three variable regions called complementarity determining regions (CDRs).

### CD8α Co-Receptor

CD4 and CD8 are transmembrane glycoproteins that serve as co-receptors for the I-cell receptor (TCR). Binding of CD4 and CD8 to MHC molecules helps stabilize weak T-cell receptor (TCR)-pMHC interactions. Meanwhile, the cytoplasric tail of CD4 and CD8 co-receptors efficiently recruit Lek, the Src kinase, to the TCR complex upon co-receptor binding to the MHC, thereby enhancing the initiation of TCR signaling and T cell activation.

To function, CD8 forms a dimer consisting of a pair of CD8 chains. The most common form of CD8 is composed of a CD8α and CD8β chain. Less-common homodimers of the CD8α chain are also expressed on some cells. A single Immunoglobulin-like domain and a long stalk region form the extracellular parts of the CD8 subunits. In its intracellular tail, the a subunit of CD8 contains the Lck-binding site while the β subunit contains a palmitoylation site.

A wildtype CD8α co-receptor (UniProt ID No. P01732) has the amino acid sequence of SEQ ID NO: 11. The extracellular domain and the transmembrane domain are shown in bolded and underlined font.

(SEQ ID NO: 11)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTFVLTLS
DERRENEGYYFCSALSNSIMYFSHFVPVFLPAKPTTTPAPRPPTPAPTIASQPLSL
RPEACRPAAGGAVHTRGLDFACDIYIWAPLAGTCGVLLLSLVITLYCNHRNRRR
VCKCPRPVVKSGDKPSLSARYV

CD4 is composed of a single chain which has four immunoglobulin domains (D1 to D4) that are exposed on the extracellular surface of the cell and a short cytoplasmic/intracellular tail. The CD4 functional motifs, including the Lek-binding site and the palmitoylation site, are within its sole intracellular domain.

A wildtype CD4 co-receptor (UniProt ID No. P01730) has the amino acid sequence of SEQ ID NO: 12. The intracellular domain is shown in bolded font. The Lck binding domain is shown in bolded and italicized font. The palmitoylation domain is shown in bolded and underlined font.

(SEQ ID NO: 12)
MNRGVPFRHLLLVLQLALLPAATQGKKVVLGKKGDTVELTCTASQKKSIQF
HWKNSNQIKILGNQGSFLTKGPSKLNDRADSRRSLWDQGNFPLIIKNLKIEDSDTYIC
EVEDQKEEVQLLVFGLTANSDTHLLQGQSLTLTLESPPGSSPSVQCRSPRGKNIQGGK
TLSVSQLELQDSGTWTCTVLQNQKKVEFKIDIVVLAFQKASSIVYKKEGEQVEFSFPL
AFTVEKLTGSGELWWQAERASSSKSWITFDLKNKEVSVKRVTQDPKLQMGKKLPL
HLTLPQALPQYAGSGNLTLALEAKTGKLHQEVNLVVMRATQLQKNLTCEVWGPTS
PKLMLSLKLENKEAKVSKREKAVWVLNPEAGMWQCLLSDSGQVLLESNIKVLPTW
STPVQPMALIVLGGVAGLLLFIGLGIFFCVRCRHRRRQAERMSQIKRLLSEKKTCQ
CPHRFQKTCSPI

### A. Protein Palmitoylation and Palmitoylation Motifs

Protein palmitoylation is a widespread lipid modification in which one or more cysteine thiols on a substrate protein are modified to form a thioester with a saturated 16-carbon fatty acid palmitoyl group. This lipid modification is readily reversible, a feature of protein palmitoylation that allows for rapid regulation of the function of many cellular proteins (e.g., see Guan and Fierke Sci China Chem. 2011 December; 54(12): 1888-1897.).

For instance, palmitoylation not only plays a role as a lipid anchor to localize proteins to the cell membrane but also in shuttling of modified proteins between cellular compartments, allowing re-localization of the protein in the cell or within different regions of the membrane. Palmitoylation motifs are small tetramer sequences comprising a cysteine residue at position 1 and position 4 of the tetramer.

In some aspects, the palmitoylation motif comprises, consists essentially of, or consists of an amino acid sequence at least 95%, 96%, 97%, 98%, 99% or 100% (or any percentage in between) identical to SEQ ID NO: 3. In some aspects, the palmitoylation motif comprises, consists essentially of, or consists of the amino acid sequence of SEQ ID NO: 3. In some aspects, the palmitoylation motif has the amino acid sequence of SEQ ID NO: 3.

In some aspects, the palmitoylation motif is encoded by a polynucleotide comprising, consisting essentially of or consisting of a nucleic acid sequence at least 95%, 96%, 97%, 98%, 99% or 100% (or any percentage in between) identical to: SEQ ID NO: 8. In some aspects, the palmitoylation motif is encoded by a polynucleotide comprising, consisting essentially of or consisting of a nucleic acid sequence of SEQ ID NO: 8.

### B. High Affinity Lck Domains

T cell signaling begins with the ligation of the T cell antigen receptor (TCR) by a cognate peptide and the phosphorylation of the receptor's immunoreceptor tyrosine-based activation motif domains by the kinase Lck.

In some aspects, the Lck binding domain (also referred to as a “High affinity Lck binding site” or a “High affinity binding domain”) comprises, consists essentially of or consists of an amino acid sequence at least 95%, 96%, 97%, 98%, 99% or 100% (or any percentage in between) identical to SEQ ID NO: 4. In some aspects, the Lck binding domain comprises, consists essentially of, or consists of the amino acid sequence of SEQ ID NO: 4. In some aspects, the Lck binding domain has the amino acid sequence of SEQ ID NO: 4.

In some aspects, the Lck binding domain is encoded by a polynucleotide comprising, consisting essentially of or consisting of a nucleic acid sequence at least 95%, 96%, 97%, 98%, 99% or 100% (or any percentage in between) identical to: SEQ ID NO: 9. In some aspects, the Lck binding domain is encoded by a polynucleotide comprising, consisting essentially of or consisting of a nucleic acid sequence of SEQ ID NO: 9.

### Exemplary Chimeric CD8α Co-Receptors

The present disclosure provides a chimeric CD8α co-receptor comprising a CD8α extracellular domain and a CD8α transmembrane domain which allows for the dimerization of the CD8α co-receptor and an intracellular domain of CD4 (with a functional motif including both a Lck-binding site and a palmitoylation site). In some aspects, the chimeric CD8α co-receptor is heterodimerized. In some aspects, the chimeric CD8α co-receptor is homodimerized. In some aspects, a homodimerized chimeric CD8α co-receptor contains two sets of the functional motif and has a higher potency at initiating TCR signaling relative to a wildtype CD8α co-receptor.

In some aspects, the CD8α extracellular and transmembrane domain comprises, consists essentially of or consists of an amino acid sequence at least 95%, 96%, 97%, 98%, 99% or 100% (or any percentage in between) identical to SEQ ID NO: 1. In some aspects, the CD8α extracellular and transmembrane domain comprises, consists essentially of, or consists of the amino acid sequence of SEQ ID NO: 1.

In some aspects, the CD8α extracellular and transmembrane domain is encoded by a polynucleotide comprising, consisting essentially of or consisting of a nucleic acid sequence at least 95%, 96%, 97%, 98%, 99% or 100% (or any percentage in between) identical to: SEQ ID NO: 6. In some aspects, the CD8α extracellular and transmembrane domain is encoded by a polynucleotide comprising, consisting essentially of or consisting of a nucleic acid sequence of SEQ ID NO: 6.

In some aspects, the CD4 intracellular domain comprises, consists essentially of or consists of an amino acid sequence at least 95%, 96%, 97%, 98%, 99% or 100% (or any percentage in between) identical to SEQ ID NO: 2. In some aspects, the CD4 intracellular domain comprises, consists essentially of, or consists of the amino acid sequence of SEQ ID NO: 2.

In some aspects, CD4 intracellular domain is encoded by a polynucleotide comprising, consisting essentially of or consisting of a nucleic acid sequence at least 95%, 96%, 97%, 98%, 99% or 100% (or any percentage in between) identical to: SEQ ID NO: 7. In some aspects, CD4 intracellular domain is encoded by a polynucleotide comprising, consisting essentially of or consisting of a nucleic acid sequence of SEQ ID NO: 7.

Exemplary chimeric CD8α co-receptors were constructed by fusing the N-terminal region of CD8α (positions 1-203 of SEQ ID NO: 11) to the C-terminal region of CD4 (positions 419-458 of SEQ ID NO: 12).

Exemplary chimeric CD8α co-receptors were constructed by fusing a wildtype N-terminal (CD8α extracellular and transmembrane domain (SEQ ID NO: 1) with a C-terminal wildtype CD4 intracellular domain (SEQ ID NO: 2). Exemplary chimeric CD8α co-receptor amino acid sequence domains and nucleic acid sequences encoding the same are shown in Tables 1 and 2.

TABLE 1
Exemplary Amino Acid Sequences of Chimeric CD8a Co-receptors

| Domain | Amino Acid Sequence | SEQ ID NO: |
|---|---|---|
| CD8α extracellular | MALPVTALLLPLALLLHAARPSQFRVSPLDRT | 1 |
| (ext) and CD8α | WNLGETVELKCQVLLSNPTSGCSWLFQPRGAA | |
| transmembrane (tm) | ASPTFLLYLSQNKPKAAEGLDTQRFSGKRLGDT | |
| domain | FVLTLSDFRRENEGYYFCSALSNSIMYFSHFVP | |
| | VFLPAKPTTTPAPRPPTPAPTIASQPLSLRPEACR | |
| | PAAGGAVHTRGLDFACDIYIWAPLAGTCGVLL | |
| | LSLVIT | |
| CD4 intracellular | CVRCRHRRRQAERMSQIKRLLSEKKTCQCPHR | 2 |
| domain | FQKTCSPI | |
| Palmitoylation motif | CVRC | 3 |
| Lck binding domain | KKTCQCPHRFQKT | 4 |
| Full Length CD8α | MALPVTALLLPLALLLHAARPSQFRVSPLDRT | 5 |
| Co-Receptor | WNLGETVELKCQVLLSNPTSGCSWLFQPRGAA | |
| [CD8α ext and tm; | ASPTFLLYLSQNKPKAAEGLDTQRFSGKRLGDT | |
| CD4 intracellular | FVLTLSDFRRENEGYYFCSALSNSIMYFSHFVP | |
| (bolded); | VFLPAKPTTTPAPRPPTPAPTIASQPLSLRPEACR | |
| palmitoylation motif | PAAGGAVHTRGLDFACDIYIWAPLAGTCGVLL | |
| (bolded and | LSLVITCVRCRHRRRQAERMSQIKRLLSEKK | |
| italicized); Lck | TCQCPHRFQKTCSPI | |
| binding domain | | |
| (bolded and | | |
| underlined)] | | |

TABLE 2
Exemplary Nucleic Acid Sequences of Chimeric CD8a Co-receptors

| Domain | Nucleic Acid Sequence | SEQ ID NO: |
|---|---|---|
| CD8α extracellular | ATGGCTCTGCCTGTGACAGCTCTGCTGCTGCC | 6 |
| and transmembrane | TCTGGCTCTGCTTCTGCATGCCGCTAGACCCA | |
| domain | GCCAGTTCAGAGTGTCCCCTCTGGACAGAAC | |
| | CTGGAACCTGGGCGAGACAGTGGAACTGAA | |
| | GTGCCAGGTGCTGCTGAGCAACCCTACAAGC | |
| | GGCTGCTCCTGGCTGTTTCAGCCTAGAGGTG | |
| | CAGCCGCCTCTCCTACCTTTCTGCTGTACCTG | |
| | AGCCAGAACAAGCCCAAGGCCGCCGAAGGA | |
| | CTGGACACCCAGAGATTCAGCGGCAAGAGAC | |
| | TGGGCGACACCTTCGTGCTGACCCTGAGCGA | |
| | CTTCAGAAGAGAGAACGAGGGCTACTACTTC | |
| | TGTAGCGCCCTGAGCAACAGCATCATGTACT | |
| | TCAGCCACTTCGTGCCCGTGTTTCTGCCCGCC | |
| | AAGCCTACAACAACCCCTGCTCCTAGACCTC | |
| | CTACACCAGCTCCTACAATCGCCAGCCAGCC | |
| | TCTGTCTCTCAGACCCGAAGCCTGTAGACCT | |
| | GCTGCTGGCGGAGCTGTGCATACCAGAGGAC | |
| | TGGATTTCGCCTGCGACATCTACATCTGGGCC | |
| | CCTCTGGCTGGAACATGTGGCGTGCTGCTGC | |
| | TGTCCCTGGTCATCACA | |
| CD4 intracellular | TGTGTGCGGTGCAGACACAGACGCAGACAGG | 7 |
| domain | CCGAGAGAATGTCCCAGATCAAGCGCCTGCT | |
| | GAGCGAGAAGAAAACCTGCCAGTGTCCTCAC | |
| | CGGTTCCAGAAAACATGCAGCCCCATCTAA | |
| Palmitoylation motif | TGTGTGCGGTGC | 8 |
| Hi-Aff Lck domain | AAGAAAACCTGCCAGTGTCCTCACCGGTTCC | 9 |
| | AGAAAACA | |
| Full Length CD8α | ATGGCTCTGCCTGTGACAGCTCTGCTGCTGCC | 10 |
| Co-Receptor | TCTGGCTCTGCTTCTGCATGCCGCTAGACCCA | |
| [CD8α ext and tm; | GCCAGTTCAGAGTGTCCCCTCTGGACAGAAC | |
| CD4 intracellular | CTGGAACCTGGGCGAGACAGTGGAACTGAA | |
| (bolded); | GTGCCAGGTGCTGCTGAGCAACCCTACAAGC | |
| palmitoylation motif | GGCTGCTCCTGGCTGTTTCAGCCTAGAGGTG | |
| (bolded and | CAGCCGCCTCTCCTACCTTTCTGCTGTACCTG | |
| italicized); Lck | AGCCAGAACAAGCCCAAGGCCGCCGAAGGA | |
| binding domain | CTGGACACCCAGAGATTCAGCGGCAAGAGAC | |
| (bolded and | TGGGCGACACCTTCGTGCTGACCCTGAGCGA | |
| underlined)] | CTTCAGAAGAGAGAACGAGGGCTACTACTTC | |
| | TGTAGCGCCCTGAGCAACAGCATCATGTACT | |
| | TCAGCCACTTCGTGCCCGTGTTTCTGCCCGCC | |
| | AAGCCTACAACAACCCCTGCTCCTAGACCTC | |
| | CTACACCAGCTCCTACAATCGCCAGCCAGCC | |
| | TCTGTCTCTCAGACCCGAAGCCTGTAGACCT | |
| | GCTGCTGGCGGAGCTGTGCATACCAGAGGAC | |
| | TGGATTTCGCCTGCGACATCTACATCTGGGCC | |
| | CCTCTGGCTGGAACATGTGGCGTGCTGCTGC | |
| | TGTCCCTGGTCATCACATGTGTGCGGTGCAG | |
| | ACACAGACGCAGACAGGCCGAGAGAATGT | |
| | CCCAGATCAAGCGCCTGCTGAGCGAGAAG | |
| | AAAACCTGCCAGTGTCCTCACCGGTTCCA | |
| | GAAAACATGCAGCCCCATCTAA | |

In some aspects, the chimeric CD8α co-receptor comprises, consists essentially of or consists of an amino acid sequence at least 95%, 96%, 97%, 98%, 99% or 100% (or any percentage in between) identical to SEQ ID NO: 5. In some aspects, the chimeric CD8α co-receptor comprises, consists essentially of, or consists of the amino acid sequence of SEQ ID NO: 5.

In some aspects, the chimeric CD8α co-receptor is encoded by a polynucleotide comprising, consisting essentially of or consisting of a nucleic acid sequence at least 95%, 96%, 97%, 98%, 99% or 100% (or any percentage in between) identical to: SEQ ID NO: 10. In some aspects, the chimeric CD8α co-receptor is encoded by a polynucleotide comprising, consisting essentially of or consisting of a nucleic acid sequence of SEQ ID NO: 10.

The chimeric CD8α co-receptor polypeptide sequence was reverse-translated and codon-optimized for human T cell expression by GeneArt® tools. The resulting nucleic acid sequence encoding the chimeric CD8α co-receptor (SEQ ID NO: 10) was cloned into a transposon vector together with TCR genes, an inducible suicide gene and a selection gene.

In certain embodiments of the disclosure, the nucleotide sequences encoding the chimeric CD8α co-receptor and the TCR are cloned into a transposon comprising bi-directional expression cassettes for expression of the chimeric CD8α co-receptor and the TCR. In some embodiments, the transposon further comprises an expression cassette for the expression of a CAR.

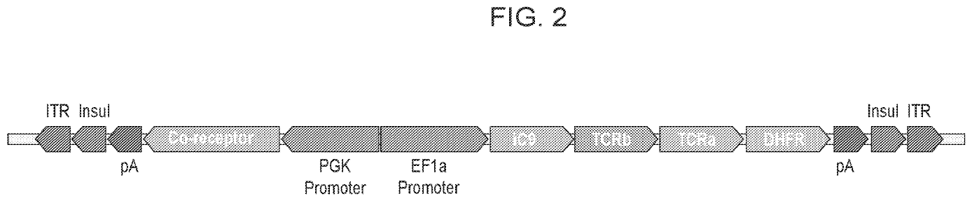

An exemplary transposon comprising a nucleotide sequence encoding a chimeric CD8α co-receptor is illustrated in FIG. 2. The transposon comprises the following nucleotide sequences in the 5′ to 3′ direction: a left inverted terminal repeat (ITR), a first insulator sequence, a first poly sequence (3′-5′), a chimeric CD8 co-receptor (3′-5′), a PGK promoter (3′-5′), an EF1a promoter, a iCAS9 safety switch, a TCR beta chain, a TCR alpha chain, a DHFR selectable marker (each of the iCAS9 safety switch, TCR beta chain, TCR alpha chain, DHFR selectable marker are separated by T2A sequences), a second polyA sequence, a second insulator sequence and a right ITR.

Compositions comprising a chimeric CD8α co-receptor of the present disclosure can be incorporated into a cell delivery composition (e.g., transposon or vector) as described in detail herein, and, optionally, can be incorporated into a cell.

### Cells and Modified Cells of the Disclosure

Cells and modified cells of the disclosure can be mammalian cells. Preferably, the cells and modified cells are human cells. Cells and modified cells of the disclosure can be immune cells. The immune cells of the disclosure can comprise lymphoid progenitor cells, T lymphocytes (T-cell), stem memory T cells (TsCM cells), central memory T cells (TCM) or stem cell-like T cells.

Modified T cells of the disclosure may be derived from modified hematopoietic stem and progenitor cells (HSPCs) or modified HSCs. Unlike traditional biologics and chemotherapeutics, the disclosed modified-T cells have the capacity to rapidly reproduce upon antigen recognition, thereby potentially obviating the need for repeat treatments. To achieve this, in some embodiments, modified-T cells not only drive an initial response, but also persist in the patient as a stable population of viable memory T cells to prevent potential relapses. Alternatively, in some aspects, when it is not desired, the modified-T cells do not persist in the patient.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of a plurality of modified T cells in the population express a chimeric CD8α co-receptor. In some aspects, the chimeric CD8α co-receptor is expressed on the cell surface as a homodimer.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of a plurality of modified T cells in the population express a TCR.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of a plurality of modified T cells in the population express a TCR and a chimeric CD8α co-receptor. In some aspects, the chimeric CD8α co-receptor is expressed on the cell surface as a homodimer.

A plurality of modified cells of the population comprise a transgene or a sequence encoding the transgene (e.g., a chimeric CD8α co-receptor and a TCR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population express the transgene or the sequence encoding the chimeric CD8α co-receptor, wherein at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express the transgene or the sequence encoding the TCR.

Compositions and methods of producing and/or expanding the immune cells or immune precursor cells (e.g., the disclosed modified T-cells) and buffers for maintaining or enhancing a level of cell viability and/or a stem-like phenotype of the immune cells or immune precursor cells (e.g., the disclosed modified T-cells) are disclosed elsewhere herein and are disclosed in more detail in U.S. Pat. No. 10,329,543 and PCT Publication No. WO 2019/173636.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogeneic cells can be stem cells or can be derived from stem cells. Allogeneic cells can be differentiated somatic cells.

### Methods of Expressing a Chimeric CD8α Co-Receptor

The disclosure provides methods of expressing a chimeric CD8α co-receptor on the surface of a cell. The method comprises (a) obtaining a cell population; (b) contacting the cell population to a composition comprising a chimeric CD8α co-receptor or a sequence encoding the chimeric CD8α co-receptor, under conditions sufficient to transfer the chimeric CD8α co-receptor across a cell membrane of at least one cell in the cell population, thereby generating a modified cell population; (c) culturing the modified cell population under conditions suitable for integration of the sequence encoding the chimeric CD8α co-receptor; and (d) expanding and/or selecting at least one cell from the modified cell population that expresses the chimeric CD8α co-receptor on the cell surface. In some embodiments the chimeric CD8α co-receptor is a homodimer. In some embodiments, the chimeric CD8α co-receptor is a heterodimer.

In some aspects, the cell population can comprise leukocytes and/or CD4+ and CD8+ leukocytes. The cell population can comprise CD4+ and CD8+ leukocytes in an optimized ratio. In some embodiments, the optimized ratio of CD4+ to CD8+ leukocytes does not naturally occur in vivo. The cell population can comprise a tumor cell.

In some aspects, the conditions sufficient to transfer the chimeric CD8α co-receptor or the sequence encoding the chimeric CD8α co-receptor, the transposon encoding the chimeric CD8α co-receptor, or the vector encoding the chimeric CD8α co-receptor across a cell membrane of at least one cell in the cell population comprises at least one of: an application of one or more pulses of electricity at a specified voltage, a buffer, and one or more supplemental factor(s). In some aspects, the conditions suitable for integration of the sequence encoding the chimeric CD8α co-receptor comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof, (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, IL1, IL3, IL4, IL5, IL6, IL8, CXCL8, IL9, IL10, IL11, IL13, IL14, IL 16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof.

The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl₂, Na₂HPO₄, NAH₂PO₄, Sodium lactobionate, Mannitol, Sodium succinate, Sodium Chloride, CINa, Glucose, Ca(NO₃)₂, Tris/HCl, K₂HPO₄, KH₂PO₄, Polyethylenimine, Poly-ethylene-glycol, Poloxamer 188, Poloxamer 181, Poloxamer 407, Poly-vinylpyrrolidone, Pop313, Crown-5, or any combination thereof. The cell medium can comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof.

The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3β) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e., second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the TCR and/or the CAR, thereby generating an expanded cell population. The antigen can be presented on the surface of a substrate. The substrate can have any form, including, but not limited to a surface, a well, a bead or a plurality thereof, and a matrix. The substrate can further comprise a paramagnetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects, wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

The present disclosure provides a cell or a population of cells wherein the cell comprises a composition comprising (a) an inducible transgene construct, comprising a sequence encoding an inducible promoter and a sequence encoding a transgene (e.g., chimeric CD8α co-receptor) and (b) an inducible transgene construct comprising a sequence encoding an inducible promoter and a sequence encoding a TCR and/or a CAR, wherein, upon integration of the construct of (a) and the construct of (b) into a genomic sequence of the cell, the chimeric CD8α co-receptor and the TCR and/or the CAR is expressed on the surface of the cell (e.g. T cell). Exemplary inducible promoters include, but are not limited to, PGK promoters and EF1a promoters. In some aspects, a PGK promoter controls the expression of the chimeric CD8α co-receptor. In some aspects, an EF1a promoter controls the expression of the TCR.

### Transposon and Vector Compositions

The present disclosure provides compositions and methods for delivering a chimeric CD8α co-receptor and/or a TCR and/or a CAR to a cell or a population of cells. Non-limiting examples of compositions for delivery of a composition of the disclosure to a cell or a population of cells include a transposon or a vector. Thus, the present disclosure provides (i) a transposon comprising a chimeric CD8α co-receptor and/or a TCR and/or a CAR; or (ii) a vector comprising a chimeric CD8α co-receptor and/or a TCR and/or a CAR.

A transposon comprising a chimeric CD8α co-receptor and/or a TCR and/or a CAR of the disclosure or a vector comprising a chimeric CD8α co-receptor and/or a TCR and/or a CAR of the disclosure can further comprise a sequence encoding an inducible proapoptotic polypeptide (e.g., iC9). Alternatively, or in addition, one transposon or one vector can comprise a chimeric CD8α co-receptor and/or a TCR and/or a CAR of the disclosure and a second transposon or second vector can comprise a sequence encoding an inducible proapoptotic polypeptide of the disclosure. Inducible proapoptotic polypeptides are described in more detail herein.

A transposon comprising a TCR and/or a CAR of the disclosure or a vector comprising a TCR and/or a CAR of the disclosure can further comprise a sequence encoding a chimeric CD8α co-receptor. Alternatively, or in addition, one transposon or one vector can comprise a TCR and/or a CAR of the disclosure and a second transposon or a second vector can comprise a sequence encoding a chimeric CD8α co-receptor. Chimeric CD8α co-receptors are described in more detail herein.

A transposon comprising a chimeric CD8α co-receptor and/or a TCR and/or a CAR of the disclosure or a vector comprising a chimeric CD8α co-receptor and/or a TCR and/or a CAR of the disclosure can further comprise a selection gene. The selection gene can encode a gene product essential for cell viability and survival. The selection gene can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2), or any combination thereof.

In a preferred aspect, the selection gene encodes a DHFR mutein enzyme. The DHFR mutein enzyme comprises, consists essentially of, or consists of the amino acid sequence of SEQ ID NO: 13. The DHFR mutein enzyme is encoded by a polynucleotide comprising, consisting essentially of or consisting of the nucleic acid sequence of SEQ ID NO: 14. The amino acid sequence of the DHFR mutein enzyme can further comprise a mutation at one or more of positions 80, 113, or 153. The amino acid sequence of the DHFR mutein enzyme can comprise one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

### Transposition Systems

The present disclosure provides a transposon or a nanotransposon for expressing a chimeric CD8α co-receptor in cells (e.g., T-cells), or co-expressing a chimeric CD8α co-receptor and a TCR in cells (e.g., T-cells). In some embodiments, the cell further comprises a CAR.

The transposon or nanotransposon of the disclosure can be a plasmid DNA transposon comprising a sequence encoding a chimeric CD8α co-receptor flanked by two cis-regulatory insulator elements. The transposon or nanotransposon can further comprise a plasmid comprising a sequence encoding a transposase. The sequence encoding the transposase may be a DNA sequence or an RNA sequence. Preferably, the sequence encoding the transposase is an mRNA sequence.

The transposon or nanotransposon of the present disclosure can be a piggyBac™ (PB) transposon. In some aspects when the transposon is a PB transposon, the transposase is a piggyBac™ (PB) transposase, a piggyBac-like (PBL) transposase or a Super piggyBac™ (SPB) transposase. In some aspects, the sequence encoding the SPB transposase is an mRNA sequence.

Nanotransposons are described in more detail in PCT/US2019/067758. Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon and insert the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon systems have no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 75%, 80%, 85%, 90%, 95%, 96%, 97%, 98%, 99% or 100% (or any percentage in between) identical to SEQ ID NO: 15.

The PB or PBL transposase can comprise or consist of an amino acid sequence having an amino acid substitution at two or more, at three or more or at each of positions 30, 165, 282, or 538 of the sequence of SEQ ID NO: 15. The transposase can be a SPB transposase that comprises or consists of the amino acid sequence of the sequence of SEQ ID NO: 15 wherein the amino acid substitution at position 30 can be a substitution of a valine (V) for an isoleucine (I), the amino acid substitution at position 165 can be a substitution of a serine (S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 75%, 80%, 85%, 90%, 95%, 96%, 97%, 98%, 99% or 100% (or any percentage in between) identical to SEQ ID NO: 16.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 15 or SEQ ID NO: 16, described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

A transposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase. In a preferred aspect, the Sleeping Beauty transposase comprises or consists of an amino acid sequence at least 75%, 80%, 85%, 90%, 95%, 96%, 97%, 98%, 99% or 100% (or any percentage in between) identical to SEQ ID NO: 17. In a preferred aspect, the hyperactive Sleeping Beauty (SB100X) transposase comprises or consists of an amino acid sequence at least 75%, 80%, 85%, 90%, 95%, 96%, 97%, 98%, 99% or 100% (or any percentage in between) identical to SEQ ID NO: 18.

A transposon of the present disclosure can be a Helraiser transposon. An exemplary Helraiser transposon includes Helibatl, which comprises or consists of a nucleic acid sequence at least 75%, 80%, 85%, 90%, 95%, 96%, 97%, 98%, 99% or 100% (or any percentage in between) identical to SEQ ID NO: 19. In some aspects, when the transposon is a Helraiser transposon, the transposase is a Helitron transposase (for example, as disclosed in WO 2019/173636). In a preferred aspect, the Helitron transposase comprises or consists of an amino acid sequence at least 75%, 80%, 85%, 90%, 95%, 96%, 97%, 98%, 99% or 100% (or any percentage in between) identical to SEQ ID NO: 20.

A transposon of the present disclosure can be a Tol2 transposon. An exemplary Tol2 transposon, including inverted repeats, subterminal sequences and the Tol2 transposase, comprises or consists of a nucleic acid sequence at least 75%, 80%, 85%, 90%, 95%, 96%, 97%, 98%, 99% or 100% (or any percentage in between) identical to SEQ ID NO: 21. In some aspects, when the transposon is a Tol2 transposon, the transposase is a Tol2 transposase (for example, as disclosed in WO 2019/173636). In a preferred aspect, the Tol2 transposase comprises or consists of an amino acid sequence at least 75%, 80%, 85%, 90%, 95%, 96%, 97%, 98%, 99% or 100% (or any percentage in between) identical to SEQ ID NO: 22.

A transposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. In a preferred aspect, the TcBuster transposase comprises or consists of an amino acid sequence at least 75%, 80%, 85%, 90%, 95%, 96%, 97%, 98%, 99% or 100% (or any percentage in between) identical to SEQ ID NO: 23. The polynucleotide encoding a TcBuster transposase can comprise or consist of a naturally occurring nucleic acid sequence or a non-naturally occurring nucleic acid sequence. In a preferred aspect, the TcBuster transposase is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 24.

In some aspects, a mutant TcBuster transposase comprises one or more sequence variations when compared to a wild type TcBuster transposase as described in more detail in PCT Publication No. WO 2019/173636 and PCT/US2019/049816.

### Vector Systems

A vector of the present disclosure can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses comprise two or more inverted terminal repeat (ITR) sequences located in cis next to a sequence encoding the chimeric CD8α co-receptor of the disclosure. Exemplary adeno-associated viruses and recombinant adeno-associated viruses include, but are not limited to, all serotypes (e.g., AAV1, AAV2, AAV3, AAV4, AAV5, AAV6, AAV7, AAV8, and AAV9). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03.

A vector of the present disclosure can be a nanoparticle. Non-limiting examples of nanoparticle vectors include nucleic acids (e.g., RNA, DNA, synthetic nucleotides, modified nucleotides or any combination thereof), amino acids (L-amino acids, D-amino acids, synthetic amino acids, modified amino acids, or any combination thereof), polymers (e.g., polymersomes), micelles, lipids (e.g., liposomes), organic molecules (e.g., carbon atoms, sheets, fibers, tubes), inorganic molecules (e.g., calcium phosphate or gold) or any combination thereof. A nanoparticle vector can be passively or actively transported across a cell membrane.

The cell delivery compositions (e.g., transposons, vectors) disclosed herein can comprise a nucleic acid encoding a therapeutic protein or therapeutic agent. Examples of therapeutic proteins include those disclosed in PCT Publication No. WO 2019/173636 and PCT/US2019/049816.

### Inducible Proapoptotic Polypeptides

The inducible proapoptotic polypeptides disclosed herein are superior to existing inducible polypeptides because the inducible proapoptotic polypeptides of the disclosure are far less immunogenic. The inducible proapoptotic polypeptides are recombinant polypeptides, and, therefore, non-naturally occurring. Further, the sequences that are recombined to produce inducible proapoptotic polypeptides that do not comprise non-human sequences that the host human immune system could recognize as “non-self” which, consequently, would induce an immune response in the subject receiving the inducible proapoptotic polypeptide, cell comprising the inducible proapoptotic polypeptide, composition comprising the inducible proapoptotic polypeptide or cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain aspects, the non-human sequence comprises a restriction site. In certain aspects, the ligand binding region can be a multimeric ligand binding region. In certain aspects, the proapoptotic peptide is a caspase polypeptide. Non-limiting examples of caspase polypeptides include caspase 1, caspase 2, caspase 3, caspase 4, caspase 5, caspase 6, caspase 7, caspase 8, caspase 9, caspase 10, caspase 11, caspase 12, and caspase 14. Preferably, the caspase polypeptide is a caspase 9 polypeptide. The caspase 9 polypeptide can be a truncated caspase 9 polypeptide. Inducible proapoptotic polypeptides can be non-naturally occurring. When the caspase is caspase 9 or a truncated caspase 9, the inducible proapoptotic polypeptides can also be referred to as an “iC9 safety switch”.

An inducible caspase polypeptide can comprise (a) a ligand binding region, (b) a linker, and (c) a caspase polypeptide, wherein the inducible proapoptotic polypeptide does not comprise a non-human sequence. In certain aspects, an inducible caspase polypeptide comprises (a) a ligand binding region, (b) a linker, and (c) a truncated caspase 9 polypeptide, wherein the inducible proapoptotic polypeptide does not comprise a non-human sequence.

The ligand binding region can comprise a FK506 binding protein 12 (FKBP12) polypeptide. The amino acid sequence of the ligand binding region that comprises a FK506 binding protein 12 (FKBP12) polypeptide can comprise a modification at position 36 of the sequence. The modification can be a substitution of valine (V) for phenylalanine (F) at position 36 (F36V). The FKBP12 polypeptide can comprise, consist essentially of or consist of, the amino acid sequence at least 75%, 80%, 85%, 90%, 95%, 96%, 97%, 98%, 99% or 100% (or any percentage in between) identical to SEQ ID NO: 25. The FKBP12 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 26.

The linker region can comprise, consist essentially of, or consist of the amino acid sequence at least 75%, 80%, 85%, 90%, 95%, 96%, 97%, 98%, 99% or 100% (or any percentage in between) identical to SEQ ID NO: 27. In some aspects, the linker region can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 28. In some aspects, the nucleic acid sequence encoding the linker does not comprise a restriction site.

The truncated caspase 9 polypeptide can comprise an amino acid sequence that does not comprise an arginine (R) at position 87 of the sequence. Alternatively, or in addition, the truncated caspase 9 polypeptide can comprise an amino acid sequence that does not comprise an alanine (A) at position 282 the sequence. The truncated caspase 9 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 29. In some aspects, the truncated caspase 9 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 30.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 31 In some aspects, the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 32.

Inducible proapoptotic polypeptides can be expressed in a cell under the transcriptional regulation of any promoter known in the art that is capable of initiating and/or regulating the expression of an inducible proapoptotic polypeptide in that cell.

Activation of inducible proapoptotic polypeptides can be accomplished through, for example, chemically induced dimerization (CID) mediated by an induction agent to produce a conditionally controlled protein or polypeptide. Proapoptotic polypeptides are not only inducible, but the induction of these polypeptides is also reversible due to the degradation of the labile dimerizing agent or administration of a monomeric competitive inhibitor.

In certain aspects when the ligand binding region comprises a FKBP12 polypeptide having a substitution of valine (V) for phenylalanine (F) at position 36 (F36V), the induction agent can comprise AP1903, a synthetic drug (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9Cl) CAS Registry Number: 195514-63-7; Molecular Formula: C78H98N4020; Molecular Weight: 1411.65)); AP20187 (CAS Registry Number: 195514-80-8 and Molecular Formula: C82H107N5O20) or an AP20187 analog, such as, for example, AP1510. As used herein, the induction agents AP20187, AP1903 and AP1510 can be used interchangeably.

Inducible proapoptotic peptides and methods of inducing these peptides are described in detail in U.S. Patent Publication No. WO 2019/0225667 and PCT Publication No. WO 2018/068022.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples and methods of preparing such sterile solutions are well known in the art, such as, but not limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. In some aspects, the amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris(tris(hydroxymethyl)aminomethane); tromethamine hydrochloride or phosphate buffers. Preferred buffers are organic acid salts, such as citrate.

Additionally, the disclosed compositions can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions. In some embodiments, the composition can be prepared for use in vaginal or rectal administration in semisolid forms, including, but not limited to, creams and suppositories. In some embodiments, the composition can be prepared for buccal or sublingual administration. Non-liming examples of preparations for buccal or sublingual administration can include tablet or capsule form. In some embodiments, the composition can be prepared for intranasal administration. Non-limiting examples of preparations for intranasal administration can include powders, nasal drops or aerosols. In some embodiments, these preparations further include certain agents. In some embodiments, the composition can be prepared for transdermal administration. Non-liming examples of preparations for transdermal administration can include gels, ointments, lotions, suspensions or patch delivery systems. In some embodiments, the transdermal administration composition further includes chemical enhancers. In some embodiments, the chemical enhancer may be dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994). In some embodiments, the suspension or patch delivery system further includes oxidizing agents that enable the application of formulations containing proteins and peptides onto the skin (WO 98/53847). In some embodiments, the transdermal administration includes the application of electric fields to create transient transport pathways (such as electroporation) or to increase the mobility of charged drugs through the skin (such as iontophoresis). In some embodiments, the transdermal administration includes the application of ultrasound, such as sonophoresis (U.S. Pat. Nos. 4,309,989 and 4,767,402). The above publications and patents are entirely incorporated herein by reference.

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols (such as polyethylene glycol), oils of vegetable origin, hydrogenated naphthalenes, and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, gas pressured needle-less injection devices as described in U.S. Pat. No. 5,851,198, and laser perforator devices as described in U.S. Pat. No. 5,839,446.

Formulations for oral administration may rely on the co-administration of (i) adjuvants (e.g., resorcinols and nonionic surfactants, such as polyoxyethylene oleyl ether and/or n-hexadecylpolyethylene ether) to artificially increase the permeability of the intestinal walls, and (ii) enzymatic inhibitors (e.g., pancreatic trypsin inhibitors, diisopropylfluorophosphate (DFF) and trasylol) to inhibit enzymatic degradation. Formulations for delivery of hydrophilic agents (including proteins and protein scaffolds) and a combination of at least two surfactants intended for oral, buccal, mucosal, nasal, pulmonary, vaginal transmembrane, or rectal administration, are described in U.S. Pat. No. 6,309,663. The active constituent compound of the solid-type dosage form for oral administration can be mixed with at least one additive including, but not limited to, sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent; lubricants, such as magnesium stearate and paraben; preserving agents, such as sorbic acid, ascorbic acid, and alpha-tocopherol; antioxidants such as cysteine; disintegrator, binders, thickeners, buffering agents, sweetening agents, flavoring agents, perfuming agents, and the like.

Tablets and pills can be further processed into enteric-coated preparations. The liquid preparations for oral administration may include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds, such as those described in U.S. Pat. Nos. 5,879,681 and 5,871,753, are known in the art and are used to deliver biologically active agents orally.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices, capable of depositing aerosolized formulations in the sinus cavity or alveoli of a patient, include metered dose inhalers, nebulizers (e.g., jet nebulizer, ultrasonic nebulizer), dry powder generators, sprayers, and the like. All such devices can be used for formulations suitable for the administration or for the dispensing of a composition or pharmaceutical composition described herein in an aerosol. Such aerosols can be comprised of either solutions (both aqueous and non-aqueous) or solid particles. Additionally, a spray, including a composition or pharmaceutical composition described herein, can be produced by forcing a suspension or solution of at least one protein scaffold through a nozzle under pressure. In a metered dose inhaler (MDI), a propellant, a composition or pharmaceutical composition described herein, and any excipients or other additives, are contained in a canister as a mixture which includes a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose, or can be aqueous or oily solutions of nasal drops. For buccal administration, excipients may include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device such as a liposome or polymeric nanoparticle, microparticle, microcapsule, or microsphere (referred to collectively as microparticles unless otherwise stated). A number of suitable devices are known in the art, including microparticles made of synthetic polymers such as polyhydroxy acids (e.g., polylactic acid, polyglycolic acid and copolymers thereof), polyorthoesters, polyanhydrides, polyphosphazenes, or natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, over periods of one week to one year from a single administration. Various slow release, depot or implant dosage forms can be utilized. For example, a dosage form can contain a pharmaceutically acceptable non-toxic salt of the compound that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like; or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, relatively insoluble salts of the disclosed compounds such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts of the disclosed compounds such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 g/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually, a dosage of an active ingredient can be about 0.1 to 100 milligrams per kilogram of body weight. Ordinarily 0.1 to 50, and preferably, 0.1 to 10 milligrams per kilogram per administration, or in sustained release form, is effective to obtain desired results.

As a non-limiting example, treatment of humans or animals can be provided as a one-time or periodic dosage of the compositions or pharmaceutical compositions disclosed herein. In some embodiments the dosage is about 0.1 to 100 mg/kg or any range, value, or fraction thereof per day on at least one of day 1-40, and/or at least one of week 1-52, and/or at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions, the active ingredient will ordinarily be present in an amount of about 0.5-99.999% by weight based on the total weight of the composition.

An effective amount can comprise an amount of about 0.001 to about 500 mg/kg (or any effective range or value therein) per single (e.g., bolus), multiple or continuous administration. In some embodiments, an effective amount achieves a serum concentration of 0.01-5000 g/ml serum concentration per single, multiple, or continuous administration, or any effective range or value therein, as done and determined using known methods, as described herein or known in the relevant arts.

In aspects where the composition to be administered to a subject in need thereof comprises the modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

### Methods of Using the Compositions of the Disclosure

The disclosure provides methods of using a disclosed composition or pharmaceutical composition for the treatment of a disease or disorder in a cell, tissue, organ, animal, or subject, as known in the art or as described herein. In some embodiments the method of using the disclosed compositions and pharmaceutical compositions comprises, e.g., administering or contacting the cell, tissue, organ, animal, or subject with a therapeutically effective amount of the composition or pharmaceutical composition. In one aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In preferred aspects, the treatment of a malignant disease or disorder comprises adoptive cell therapy. For example, in one aspect, the disclosure provides modified cells that express at least the chimeric CD8α co-receptor and/or TCR and/or CAR that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier. Modified cells can be formulated in a pharmaceutically acceptable carrier at a prescribed density with one or more reagents to inhibit further expansion and/or to prevent cell death.

In some aspects, the treatment of a malignant disease or disorder can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering at least one chemotherapeutic agent (e.g., an alkylating agent, an a mitotic inhibitor, a radiopharmaceutical), before, concurrently, and/or after the administration of a composition or pharmaceutical composition disclosed herein.

In some aspects, the subject does not develop graft vs. host (GvH) and/or host vs. graft (HvG) following administration. In one aspect, the administration is systemic. Systemic administration can be any means known in the art and described in detail herein. Preferably, systemic administration is by an intravenous injection or an intravenous infusion. In one aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between, that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising at least a chimeric CD8α co-receptor and/or TCR and/or CAR that specifically binds to an antigen on a tumor cell. In aspects wherein the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises a step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some embodiments, oligonucleotide probes that selectively hybridize under stringent conditions to the polynucleotides of the present invention are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be affected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or groups of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell during purification or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Agl4, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Agl4 cells (ATCC Accession Number CRL-1851). In a preferred aspect, the recombinant cell is a P3X63Ab8.653 or an SP2/0-Agl4 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), or at least one human promoter); an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof, (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### Examples

### Example 1—Preparation and Storage of Allogeneic TCR T Cells Co-Expressing Chimeric CD8α Co-Receptors

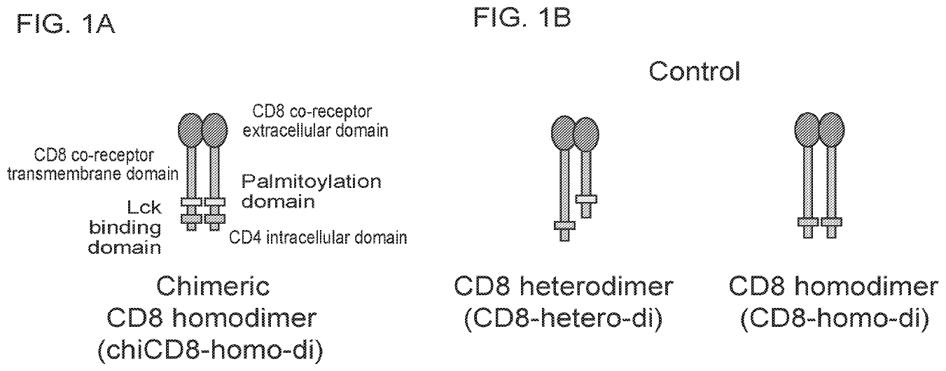

FIG. 1A shows a schematic representation of an exemplary chimeric CD8α homodimer co-receptor of the present disclosure. The chimeric CD8α homodimer co-receptor comprises a truncated CD8α co-receptor comprising a CD8α co-receptor extracellular domain and CD8α co-receptor transmembrane domain; and a CD4 intracellular domain comprising a palmitoylation domain and a high-affinity Lck binding domain. FIG. 2 shows a schematic depicting the orientation of domains of a DNA piggyBac nanotransposon containing sequence encoding an exemplary chimeric CD8α co-receptor of the disclosure and a TCR alpha and a TCR beta chains of a TCR. The chimeric CD8α co-receptor is operably linked to, and expression is controlled by, the PGK promoter. The iCAS9 safety switch, TCR beta chain, TCR alpha chain, and DHFR selectable marker are operably linked to, and expression is controlled by, the EF1a promoter. The transposon allows for the co-expression of a TCR and a chimeric CD8α co-receptor from a single construct.

Human pan T-cells were collected from healthy human donors by apheresis. Allogeneic TCR-T cells were produced from T-cells collected using an automated, positive immunomagnetic enrichment protocol for CD4 and CD8 T cells (CliniMACS CD4 MicroBeads and CliniMACS CD8 MicroBeads, respectively) using a CliniMACS Prodigy Instrument (Miltenyi Biotec). Enriched CD4 and CD8 T cells were cryopreserved in a mixture of Hank's balanced salt solution (HBSS, 40%), human serum albumin (HSA, 50%), and dimethyl sulfoxide (DMSO, 10%) prior to freezing and storage in a liquid nitrogen vapor phase.

Frozen, enriched CD4 and CD8 T cells were thawed in T cell expansion medium (ImmunoCult™-XF T Cell Expansion Medium, StemCell) and recovered overnight at 37° C. in the presence of 5% CO2. T cells were resuspended in supplemented P3 Primary Cell Nucleofector solution prior to electroporation. The transposon of FIG. 2 was introduced into enriched T cells via electroporation. Each electroporation reaction comprised: mRNAs encoding super piggyBac (SPB) transposase; Cas-CLOVER (CC); synthetic, chemically modified gRNAs targeting the TRAC, TRBC1/2 and B2M genes; as well as a DNA piggyBac nanotransposon plasmid encoding the chimeric CD8α co-receptor, the TCR alpha and beta chains of a TCR directed against the target NY-ESO (NYESO_TCR_1G4), a dihydrofolate reductase (DHFR) selection cassette, and the inducible Caspase-9 (iCasp) safety switch. Contents were preloaded into 100 μl of Nucleocuvette vessel. T cell suspensions were added, and vessels were electroporated using a 4D-Nucleofector system according to the manufacturer's instructions.

Cells were recovered post-electroporation in 20 ml T cell expansion medium (ImmunoCult™-XF T Cell Expansion Medium, StemCell) for a total culture of multiple G-Rex 6M vessels (Wilson Wolf Corporation). Cells were incubated overnight. After electroporation and extended resting time for recovery, cells were activated through the TCR in the absence of exogenous cytokines. Following activation, fresh culture medium supplemented with methotrexate (MTX) but lacking exogenous cytokines was added to select for DHFR positive T-cells. The cell culture was maintained and routinely supplemented with fresh culture medium throughout the culture period to ensure sufficient cell expansion. Cell harvest was performed after the cultured cells exited exponential growth phase and entered the stationary phase.

The harvested cells were stained with biotinylated CliniMACS® TCRα/β Antibody (Miltenyi) and incubated with CliniMACS® Anti-Biotin GMP MicroBeads (Miltenyi) for 30 min. Labeled cells were then loaded into LS column (Miltenyi) and subjected to immunomagnetic depletion. Since transgenic TCR were engineered to mask the epitope where CliniMACS® TCRα/β Antibody binds, cells expressing endogenous TCR were labeled and depleted. The endogenous TCR-depleted cells were cryopreserved in a mixture of HBSS (40%), HSA (50%), and DMSO (10%) before storage in liquid nitrogen vapor phase.

Un-transposed pan-T cells lacking transposon DNA during electroporation and no MTX selection were used as controls.

### Example 2—Co-Expression of TCR and Chimeric CD8α Co-Receptor Enhances CD4+ and CD8+ MHC1-Mediated Cytotoxicity

Allogeneic TCR-T cells prepared in Example 1 were thawed and resuspended in RPMI 1640 medium (Gibco), supplemented with 10% FBS (Sigma-Aldrich) and 2 mM Glutamax (Gibco) (RPMI-10). Post-thaw, all cells were stained with orange/propidium iodide (Logos Biosystem), so that viable nucleated cells would be fluoresce green and nonviable nucleated cells would fluoresce red. The live cells were counted by using a LunaFL Dual Fluorescence Cell Counter (Logos Biosystems).

A37-GFP melanoma cells (HLA-A2+/NY-ESO-1+/GFP+) were used as target cells in T-cell cytotoxicity assays. The target cells were thawed in DMEM-10 medium. DMEM-10 medium was prepared by mixing DMEM medium (Gibco) with 10% FBS (Sigma-Aldrich), and the cells were incubated at 37° C. and 5% CO2. After a 72 h culture period, fully confluent cultures were counted, pelleted, and resuspended in DMEM-10 medium at a concentration of 5×10⁴ cells/ml. A 100 μl aliquot of 5×10³ cells targets cells was plated in each well of a Corning Poly-D-lysine treated flat bottom plate (Corning). Each plate was incubated at 37° C. and 5% CO2 overnight.

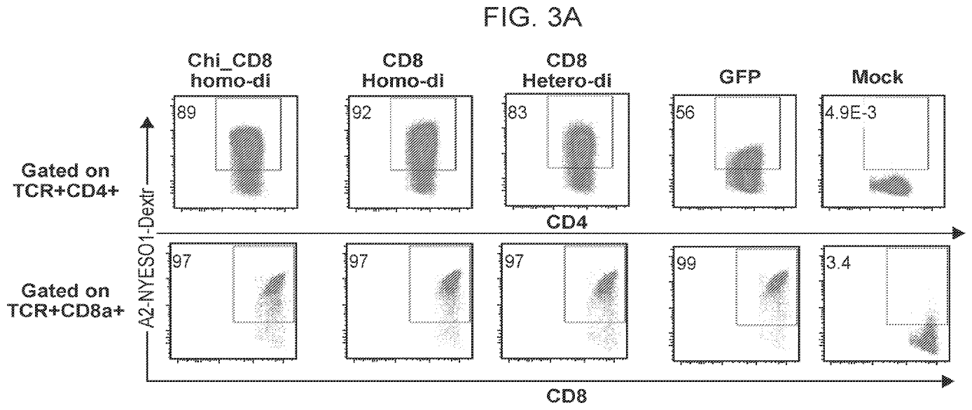

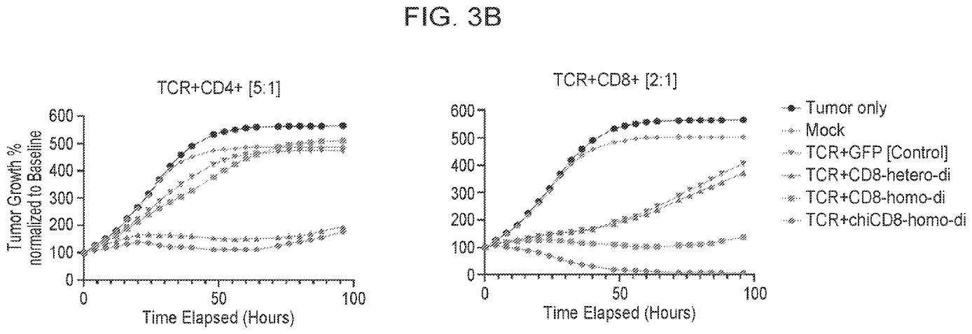

Allogeneic TCR-T cells were stained using an antibody cocktail consisting of: APC-Vb-13.1 antibody (Miltenyi), a BV785-hCD4 antibody (Biolegend), a APC-Cy7-hCD8 antibody (Biolegend), a BV421-hTCR (Biolegend), a PE-A2:NYESO Dextramer (Immudex) and a Live/Dead Fixed Aqua Dead cell stain Reagent (ThermoFisher). The stained cells were analyzed for Dextramer+frequency in a BD LSRFortessa™ Cell Analyzer or sorted to isolate Live/CD4+/Vb-13.1+ and Live/CD8+/Vb-13.1+population using a Sony SH800S sorter in accordance with the manufacturer's instructions. FIG. 3A shows a series of flow cytometry contour plots depicting the sorting of stained cells.

Sorted TCR-T cells were pelleted and resuspended in RPMI-10 at a concentration of 2.5×10⁵ cells/mL for Live/CD4+/Vb-13.1+ cells and 1×10⁵ cells/mL for Live/CD8+/Vb-13.1+ cells. A 100 μl aliquot of sorted TCR-T cells was plated in triplicate into a Corning poly-D-lysine treated 96 wells plate containing cultured A375 cells. TCR+CD4+ T cells were incubated with A375 target cells at a ratio of 5:1. TCR+CD8+ T cells were cultured with A375 target cells at a ratio of 2:1. Each plate was incubated at 37° C. and 5% CO2 for 96±2 h. TCR-T mediated cytotoxicity was analyzed by tracking GFP+A375 target cell growth using a Sartorius Incucyte system. Tumor growth percentage was measured for 100 hours. All data were exported to Microsoft Excel and plotted using GraphPad Prism software. FIG. 3B shows the cytotoxicity of T cells co-expressing TCR and chimeric CD8α co-receptor, TCR homodimer or TCR heterodimer compared to GFP and mock controls.

As shown in FIG. 3B, co-expression of the chimeric CD8α co-receptor (“TCR+chiCD8-homo-di”) resulted in enhanced TCR-mediated cytotoxicity (reduced tumor growth) compared to a wild type CD8α homodimer co-receptor (“TCR+CD8-homo-di”), a heterodimeric CD8α co-receptor (“TCR+CD8-h-di”) or controls (“TCR+GFP”; “mock”) in both CD8+ and CD4+ T cells, with a greater effect observed for CD4+ T cells.

What is claimed is:

1. A chimeric CD8α co-receptor, comprising:
a) a truncated CD8α co-receptor, comprising a CD8α co-receptor extracellular domain and CD8α co-receptor transmembrane domain; and
b) a CD4 intracellular domain comprising a palmitoylation motif and a Lck binding domain;
wherein the CD4 intracellular domain is fused in frame to the C-terminus of the truncated CD8α co-receptor.

2. The chimeric CD8α co-receptor of claim 1, wherein the truncated CD8α co-receptor comprises the amino acid sequence of SEQ ID NO: 1.

3. The chimeric CD8α co-receptor of claim 1, wherein the palmitoylation motif comprises the amino acid sequence of SEQ ID NO: 3.

4. The chimeric CD8α co-receptor of claim 1, wherein the Lck binding domain comprises the amino acid sequence of SEQ ID NO: 4.

5. The chimeric CD8α co-receptor of claim 1, wherein the CD4 intracellular domain comprising the palmitoylation motif and the Lck binding domain comprises the amino acid sequence of SEQ ID NO: 2.

6. The chimeric CD8α co-receptor of claim 1, wherein the chimeric CD8α co-receptor comprises the amino acid sequence of SEQ ID NO: 5.

7. A polynucleotide comprising a nucleic acid sequence encoding the chimeric CD8α co-receptor of any one of claims 1-6.

8. The polynucleotide of claim 7, wherein the polynucleotide is an mRNA molecule.

9. The polynucleotide of claim 7, wherein the polynucleotide is a DNA molecule.

10. The polynucleotide of claim 9, further comprising a promoter sequence operably linked with the DNA molecule to produce at least one mRNA molecule encoding the chimeric CD8α co-receptor in a cell.

11. A cell comprising the polynucleotide of any one of claims 7-10.

12. The cell of claim 11, wherein the cell expresses the chimeric CD8α co-receptor.

13. The cell of claim 11, wherein the cell further expresses a T-cell receptor (TCR).

14. The cell of any one of claims 11-13, wherein the cell is a T-cell.

15. The cell of claim 14, wherein the chimeric CD8α co-receptor is expressed as a homodimer on the cell membrane of the T-cell.

16. A pharmaceutical composition comprising the cell of any one of claims 11-15 and at least one pharmaceutically acceptable carrier or medicament.

17. A method of stimulating T cell Receptor (TCR)-mediated cytotoxicity of a population of T-cells in a subject in need thereof, comprising:
a) introducing to a population of T cells, a polynucleotide encoding a TCR and a polynucleotide encoding the chimeric CD8α co-receptor of any one of claims 1-6,
wherein the plurality of T-cells in the population of T-cells co-expresses the TCR and the chimeric CD8α co-receptor on the cell membrane of the T-cell, and
wherein the chimeric CD8α co-receptor is expressed as a homodimer; and
b) administering the population of T-cells to the subject in need thereof,
wherein the population of T-cells expressing the TCR and the chimeric CD8α co-receptor has a greater level of cytotoxicity in comparison to a population of T-cells expressing only the TCR.

18. A chimeric CD8α co-receptor, comprising:
a) a truncated CD8α co-receptor comprising the amino acid sequence of SEQ ID NO: 1; and
b) a CD4 intracellular domain comprising a palmitoylation motif comprising the amino acid sequence of SEQ ID NO: 3 and a Lck binding domain comprising the amino acid sequence of SEQ ID NO: 4;
wherein the CD4 intracellular domain is fused in frame to the C-terminus of the truncated CD8α co-receptor.

19. A chimeric CD8α co-receptor, comprising:
a) a truncated CD8α co-receptor comprising the amino acid sequence of SEQ ID NO: 1; and
b) a CD4 intracellular domain comprising the amino acid sequence of SEQ ID NO: 2;
wherein the CD4 intracellular domain is fused in frame to the C-terminus of the truncated CD8α co-receptor.


SEQUENCE LISTING (32 sequences)

SEQ ID NO: 1 - protein, 203 aa, synthetic construct
  REGION 1..203: note = CD8a coreceptor ext and tm domain
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVIT

SEQ ID NO: 2 - protein, 40 aa, synthetic construct
  REGION 1..40: note = CD4 intracellular domain
CVRCRHRRRQAERMSQIKRLLSEKKTCQCPHRFQKTCSPI

SEQ ID NO: 3 - protein, 4 aa, synthetic construct
  REGION 1..4: note = palmitoylation motif
CVRC

SEQ ID NO: 4 - protein, 13 aa, synthetic construct
  REGION 1..13: note = Lck binding domain
KKTCQCPHRFQKT

SEQ ID NO: 5 - protein, 243 aa, synthetic construct
  REGION 1..243: note = full length chimeric CD8a coreceptor
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITCVRCRHRRRQAERMSQIKRLLSEKKTCQCPHRFQKTCSPI

SEQ ID NO: 6 - DNA, 609 bp, synthetic construct
  misc_feature 1..609: note = CD8a coreceptor ext and tm domain - NT
  source 1..609: mol_type = other DNA
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaaccctacaagcggctgctcctggctgtttcagcctagaggtgcagccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgtagcgccctgagcaacagcatcatgtacttcagccacttcgtgcccgtgtttctgcccgccaagcctacaacaacccctgctcctagacctcctacaccagctcctacaatcgccagccagcctctgtctctcagacccgaagcctgtagacctgctgctggcggagctgtgcataccagaggactggatttcgcctgcgacatctacatctgggcccctctggctggaacatgtggcgtgctgctgctgtccctggtcatcaca

SEQ ID NO: 7 - DNA, 123 bp, synthetic construct
  misc_feature 1..123: note = CD4 intracellular domain - NT
  source 1..123: mol_type = other DNA
tgtgtgcggtgcagacacagacgcagacaggccgagagaatgtcccagatcaagcgcctgctgagcgagaagaaaacctgccagtgtcctcaccggttccagaaaacatgcagccccatctaa

SEQ ID NO: 8 - DNA, 12 bp, synthetic construct
  misc_feature 1..12: note = palmitoylation motif - NT
  source 1..12: mol_type = other DNA
tgtgtgcggtgc

SEQ ID NO: 9 - DNA, 39 bp, synthetic construct
  misc_feature 1..39: note = Lck binding domain - NT
  source 1..39: mol_type = other DNA
aagaaaacctgccagtgtcctcaccggttccagaaaaca

SEQ ID NO: 10 - DNA, 732 bp, synthetic construct
  misc_feature 1..732: note = full length chimeric CD8a coreceptor - NT
  source 1..732: mol_type = other DNA
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaaccctacaagcggctgctcctggctgtttcagcctagaggtgcagccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgtagcgccctgagcaacagcatcatgtacttcagccacttcgtgcccgtgtttctgcccgccaagcctacaacaacccctgctcctagacctcctacaccagctcctacaatcgccagccagcctctgtctctcagacccgaagcctgtagacctgctgctggcggagctgtgcataccagaggactggatttcgcctgcgacatctacatctgggcccctctggctggaacatgtggcgtgctgctgctgtccctggtcatcacatgtgtgcggtgcagacacagacgcagacaggccgagagaatgtcccagatcaagcgcctgctgagcgagaagaaaacctgccagtgtcctcaccggttccagaaaacatgcagccccatctaa

SEQ ID NO: 11 - protein, 235 aa, synthetic construct
  REGION 1..235: note = wildtype CD8a coreceptor
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 12 - protein, 458 aa, synthetic construct
  REGION 1..458: note = wildtype CD4 co-receptor
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 13 - protein, 187 aa, synthetic construct
  REGION 1..187: note = DHFR gene
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 14 - DNA, 561 bp, synthetic construct
  misc_feature 1..561: note = DHFR gene - NT
  source 1..561: mol_type = other DNA
atggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 15 - protein, 594 aa, synthetic construct
  REGION 1..594: note = SPB transposase
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 16 - protein, 594 aa, synthetic construct
  REGION 1..594: note = SPBtransposase 2
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 17 - protein, 340 aa, synthetic construct
  REGION 1..340: note = Sleeping Beauty transposase
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 18 - protein, 340 aa, synthetic construct
  REGION 1..340: note = hyperactive Sleeping Beauty transposase
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 19 - DNA, 5296 bp, synthetic construct
  misc_feature 1..5296: note = Helraiser transposon - NT
  source 1..5296: mol_type = other DNA
tcctatataataaaagagaaacatgcaaattgaccatccctccgctacgctcaagccacgcccaccagccaatcagaagtgactatgcaaattaacccaacaaagatggcagttaaatttgcatacgcaggtgtcaagcgccccaggaggcaacggcggccgcgggctcccaggaccttcgctggccccgggaggcgaggccggccgcgcctagccacacccgcgggctcccgggaccttcgccagcagagagcagagcgggagagcgggcggagagcgggaggtttggaggacttggcagagcaggaggccgctggacatagagcagagcgagagagagggtggcttggagggcgtggctccctctgtcaccccagcttcctcatcacagctgtggaaactgacagcagggaggaggaagtcccacccccacagaatcagccagaatcagccgttggtcagacagctctcagcggcctgacagccaggactctcattcacctgcatctcagaccgtgacagtagagaggtgggactatgtctaaagaacaactgttgatacaacgtagctctgcagccgaaagatgccggcgttatcgacagaaaatgtctgcagagcaacgtgcgtctgatcttgaaagaaggcggcgcctgcaacagaatgtatctgaagagcagctactggaaaaacgtcgctctgaagccgaaaaacagcggcgtcatcgacagaaaatgtctaaagaccaacgtgcctttgaagttgaaagaaggcggtggcgacgacagaatatgtctagagaacagtcatcaacaagtactaccaataccggtaggaactgccttctcagcaaaaatggagtacatgaggatgcaattctcgaacatagttgtggtggaatgactgttcgatgtgaattttgcctatcactaaatttctctgatgaaaaaccatccgatgggaaatttactcgatgttgtagcaaagggaaagtctgtccaaatgatatacattttccagattacccggcatatttaaaaagattaatgacaaacgaagattctgacagtaaaaatttcatggaaaatattcgttccataaatagttcttttgcttttgcttccatgggtgcaaatattgcatcgccatcaggatatgggccatactgttttagaatacacggacaagtttatcaccgtactggaactttacatccttcggatggtgtttctcggaagtttgctcaactctatattttggatacagccgaagctacaagtaaaagattagcaatgccagaaaaccagggctgctcagaaagactcatgatcaacatcaacaacctcatgcatgaaataaatgaattaacaaaatcgtacaagatgctacatgaggtagaaaaggaagcccaatctgaagcagcagcaaaaggtattgctcccacagaagtaacaatggcgattaaatacgatcgtaacagtgacccaggtagatataattctccccgtgtaaccgaggttgctgtcatattcagaaacgaagatggagaacctccttttgaaagggacttgctcattcattgtaaaccagatcccaataatccaaatgccactaaaatgaaacaaatcagtatcctgtttcctacattagatgcaatgacatatcctattctttttccacatggtgaaaaaggctggggaacagatattgcattaagactcagagacaacagtgtaatcgacaataatactagacaaaatgtaaggacacgagtcacacaaatgcagtattatggatttcatctctctgtgcgggacacgttcaatcctattttaaatgcaggaaaattaactcaacagtttattgtggattcatattcaaaaatggaggccaatcggataaatttcatcaaagcaaaccaatctaagttgagagttgaaaaatatagtggtttgatggattatctcaaatctagatctgaaaatgacaatgtgccgattggtaaaatgataatacttccatcatcttttgagggtagtcccagaaatatgcagcagcgatatcaggatgctatggcaattgtaacgaagtatggcaagcccgatttattcataaccatgacatgcaaccccaaatgggcagatattacaaacaatttacaacgctggcaaaaagttgaaaacagacctgacttggtagccagagtttttaatattaagctgaatgctcttttaaatgatatatgtaaattccatttatttggcaaagtaatagctaaaattcatgtcattgaatttcagaaacgcggactgcctcacgctcacatattattgatattagatagtgagtccaaattacgttcagaagatgacattgaccgtatagttaaggcagaaattccagatgaagaccagtgtcctcgactttttcaaattgtaaaatcaaatatggtacatggaccatgtggaatacaaaatccaaatagtccatgtatggaaaatggaaaatgttcaaagggatatccaaaagaatttcaaaatgcgaccattggaaatattgatggatatcccaaatacaaacgaagatctggtagcaccatgtctattggaaataaagttgtcgataacacttggattgtcccttataacccgtatttgtgccttaaatataactgtcatataaatgttgaagtctgtgcatcaattaaaagtgtcaaatatttatttaaatacatctataaagggcacgattgtgcaaatattcaaatttctgaaaaaaatattatcaatcatgacgaagtacaggacttcattgactccaggtatgtgagcgctcctgaggctgtttggagactttttgcaatgcgaatgcatgaccaatctcatgcaatcacaagattagctattcatttgccaaatgatcagaatttgtattttcataccgatgattttgctgaagttttagatagggctaaaaggcataactcgactttgatggcttggttcttattgaatagagaagattctgatgcacgtaattattattattgggagattccacagcattatgtgtttaataattctttgtggacaaaacgccgaaagggtgggaataaagtattaggtagactgttcactgtgagctttagagaaccagaacgatattaccttagacttttgcttctgcatgtaaaaggtgcgataagttttgaggatctgcgaactgtaggaggtgtaacttatgatacatttcatgaagctgctaaacaccgaggattattacttgatgacactatctggaaagatacgattgacgatgcaatcatccttaatatgcccaaacaactacggcaactttttgcatatatatgtgtgtttggatgtccttctgctgcagacaaattatgggatgagaataaatctcattttattgaagatttctgttggaaattacaccgaagagaaggtgcctgtgtgaactgtgaaatgcatgcccttaacgaaattcaggaggtattcacattgcatggaatgaaatgttcacatttcaaacttccggactatcctttattaatgaatgcaaatacatgtgatcaattgtacgagcaacaacaggcagaggttttgataaattctctgaatgatgaacagttggcagcctttcagactataacttcagccatcgaagatcaaactgtacaccccaaatgctttttcttggatggtccaggtggtagtggaaaaacatatctgtataaagttttaacacattatattagaggtcgtggtggtactgttttacccacagcatctacaggaattgctgcaaatttacttcttggtggaagaacctttcattcccaatataaattaccaattccattaaatgaaacttcaatttctagactcgatataaagagtgaagttgctaaaaccattaaaaaggcccaacttctcattattgatgaatgcaccatggcatccagtcatgctataaacgccatagatagattactaagagaaattatgaatttgaatgttgcatttggtgggaaagttctccttctcggaggggattttcgacaatgtctcagtattgtaccacatgctatgcgatcggccatagtacaaacgagtttaaagtactgtaatgtttggggatgtttcagaaagttgtctcttaaaacaaatatgagatcagaggattctgcttatagtgaatggttagtaaaacttggagatggcaaacttgatagcagttttcatttaggaatggatattattgaaatcccccatgaaatgatttgtaacggatctattattgaagctacctttggaaatagtatatctatagataatattaaaaatatatctaaacgtgcaattctttgtccaaaaaatgagcatgttcaaaaattaaatgaagaaattttggatatacttgatggagattttcacacatatttgagtgatgattccattgattcaacagatgatgctgaaaaggaaaattttcccatcgaatttcttaatagtattactccttcgggaatgccgtgtcataaattaaaattgaaagtgggtgcaatcatcatgctattgagaaatcttaatagtaaatggggtctttgtaatggtactagatttattatcaaaagattacgacctaacattatcgaagctgaagtattaacaggatctgcagagggagaggttgttctgattccaagaattgatttgtccccatctgacactggcctcccatttaaattaattcgaagacagtttcccgtgatgccagcatttgcgatgactattaataaatcacaaggacaaactctagacagagtaggaatattcctacctgaacccgttttcgcacatggtcagttatatgttgctttctctcgagttcgaagagcatgtgacgttaaagttaaagttgtaaatacttcatcacaagggaaattagtcaagcactctgaaagtgtttttactcttaatgtggtatacagggagatattagaataagtttaatcactttatcagtcattgtttgcatcaatgttgtttttatatcatgtttttgttgtttttatatcatgtctttgttgttgttatatcatgttgttattgtttatttattaataaatttatgtattattttcatatacattttactcatttcctttcatctctcacacttctattatagagaaagggcaaatagcaatattaaaatatttcctctaattaattccctttcaatgtgcacgaatttcgtgcaccgggccactag

SEQ ID NO: 20 - protein, 1496 aa, synthetic construct
  REGION 1..1496: note = Helitron transposase
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 21 - DNA, 4682 bp, synthetic construct
  misc_feature 1..4682: note = Tol2 transposon - NT
  source 1..4682: mol_type = other DNA
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttaaatgttatttattctgcctatgaaaatcgttttcacattatatgaaattggtcagacatgttcattggtcctttggaagtgacgtcatgtcacatctattaccacaatgcacagcaccttgacctggaaattagggaaattataacagtcaatcagtggaagaaaatggaggaagtatgtgattcatcagcagctgcgagcagcacagtccaaaatcagccacaggatcaagagcacccgtggccgtatcttcgcgaattcttttctttaagtggtgtaaataaagattcattcaagatgaaatgtgtcctctgtctcccgcttaataaagaaatatcggccttcaaaagttcgccatcaaacctaaggaagcatattgaggtaagtacattaagtattttgttttactgatagtttttttttttttttttttttttttttttgggtgtgcatgttttgacgttgatggcgcgccttttatatgtgtagtaggcctattttcactaatgcatgcgattgacaatataaggctcacgtaataaaatgctaaaatgcatttgtaattggtaacgttaggtccacgggaaatttggcgcctattgcagctttgaataatcattatcattccgtgctctcattgtgtttgaattcatgcaaaacacaagaaaaccaagcgagaaatttttttccaaacatgttgtattgtcaaaacggtaacactttacaatgaggttgattagttcatgtattaactaacattaaataaccatgagcaatacatttgttactgtatctgttaatctttgttaacgttagttaatagaaatacagatgttcattgtttgttcatgttagttcacagtgcattaactaatgttaacaagatataaagtattagtaaatgttgaaattaacatgtatacgtgcagttcattattagttcatgttaactaatgtagttaactaacgaaccttattgtaaaagtgttaccatcaaaactaatgtaatgaaatcaattcaccctgtcatgtcagccttacagtcctgtgtttttgtcaatataatcagaaataaaattaatgtttgattgtcactaaatgctactgtatttctaaaatcaacaagtatttaacattataaagtgtgcaattggctgcaaatgtcagttttattaaagggttagttcacccaaaaatgaaaataatgtcattaatgactcgccctcatgtcgttccaagcccgtaagacctccgttcatcttcagaacacagtttaagatattttagatttagtccgagagctttctgtgcctccattgagaatgtatgtacggtatactgtccatgtccagaaaggtaataaaaacatcaaagtagtccatgtgacatcagtgggttagttagaattttttgaagcatcgaatacattttggtccaaaaataacaaaacctacgactttattcggcattgtattctcttccgggtctgttgtcaatccgcgttcacgacttcgcagtgacgctacaatgctgaataaagtcgtaggttttgttatttttggaccaaaatgtattttcgatgcttcaaataattctacctaacccactgatgtcacatggactactttgatgtttttattacctttctggacatggacagtataccgtacatacattttcagtggagggacagaaagctctcggactaaatctaaaatatcttaaactgtgttccgaagatgaacggaggtgttacgggcttggaacgacatgagggtgagtcattaatgacatcttttcatttttgggtgaactaaccctttaatgctgtaatcagagagtgtatgtgtaattgttacatttattgcatacaatataaatatttatttgttgtttttacagagaatgcacccaaattacctcaaaaactactctaaattgacagcacagaagagaaagatcgggacctccacccatgcttccagcagtaagcaactgaaagttgactcagttttcccagtcaaacatgtgtctccagtcactgtgaacaaagctatattaaggtacatcattcaaggacttcatcctttcagcactgttgatctgccatcatttaaagagctgattagtacactgcagcctggcatttctgtcattacaaggcctactttacgctccaagatagctgaagctgctctgatcatgaaacagaaagtgactgctgccatgagtgaagttgaatggattgcaaccacaacggattgttggactgcacgtagaaagtcattcattggtgtaactgctcactggatcaaccctggaagtcttgaaagacattccgctgcacttgcctgcaaaagattaatgggctctcatacttttgaggtactggccagtgccatgaatgatatccactcagagtatgaaatacgtgacaaggttgtttgcacaaccacagacagtggttccaactttatgaaggctttcagagtttttggtgtggaaaacaatgatatcgagactgaggcaagaaggtgtgaaagtgatgacactgattctgaaggctgtggtgagggaagtgatggtgtggaattccaagatgcctcacgagtcctggaccaagacgatggcttcgaattccagctaccaaaacatcaaaagtgtgcctgtcacttacttaacctagtctcaagcgttgatgcccaaaaagctctctcaaatgaacactacaagaaactctacagatctgtctttggcaaatgccaagctttatggaataaaagcagccgatcggctctagcagctgaagctgttgaatcagaaagccggcttcagcttttaaggccaaaccaaacgcggtggaattcaacttttatggctgttgacagaattcttcaaatttgcaaagaagcaggagaaggcgcacttcggaatatatgcacctctcttgaggttccaatgtaagtgtttttcccctctatcgatgtaaacaaatgtgggttgtttttgtttaatactctttgattatgctgatttctcctgtaggtttaatccagcagaaatgctgttcttgacagagtgggccaacacaatgcgtccagttgcaaaagtactcgacatcttgcaagcggaaacgaatacacagctggggtggctgctgcctagtgtccatcagttaagcttgaaacttcagcgactccaccattctctcaggtactgtgacccacttgtggatgccctacaacaaggaatccaaacacgattcaagcatatgtttgaagatcctgagatcatagcagctgccatccttctccctaaatttcggacctcttggacaaatgatgaaaccatcataaaacgaggtaaatgaatgcaagcaacatacacttgacgaattctaatctgggcaacctttgagccataccaaaattattcttttatttatttatttttgcactttttaggaatgttatatcccatctttggctgtgatctcaatatgaatattgatgtaaagtattcttgcagcaggttgtagttatccctcagtgtttcttgaaaccaaactcatatgtatcatatgtggtttggaaatgcagttagattttatgctaaaataagggatttgcatgattttagatgtagatgactgcacgtaaatgtagttaatgacaaaatccataaaatttgttcccagtcagaagcccctcaaccaaacttttctttgtgtctgctcactgtgcttgtaggcatggactacatcagagtgcatctggagcctttggaccacaagaaggaattggccaacagttcatctgatgatgaagattttttcgcttctttgaaaccgacaacacatgaagccagcaaagagttggatggatatctggcctgtgtttcagacaccagggagtctctgctcacgtttcctgctatttgcagcctctctatcaagactaatacacctcttcccgcatcggctgcctgtgagaggcttttcagcactgcaggattgcttttcagccccaaaagagctaggcttgacactaacaattttgagaatcagcttctactgaagttaaatctgaggttttacaactttgagtagcgtgtactggcattagattgtctgtcttatagtttgataattaaatacaaacagttctaaagcaggataaaaccttgtatgcatttcatttaatgttttttgagattaaaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 22 - protein, 649 aa, synthetic construct
  REGION 1..649: note = Tol2 transposon
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 23 - protein, 636 aa, synthetic construct
  REGION 1..636: note = TcBuster transposase
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 24 - DNA, 1911 bp, synthetic construct
  misc_feature 1..1911: note = TcBuster transposase - NT
  source 1..1911: mol_type = other DNA
atgatgttgaattggctgaaaagtggaaagcttgaaagtcaatcacaggaacagagttcctgctaccttgagaactctaactgcctgccaccaacgctcgattctacagatattatcggtgaagagaacaaagctggtaccacctctcgcaagaagcggaaatatgacgaggactatctgaacttcggttttacatggactggcgacaaggatgagcccaacggactttgtgtgatttgcgagcaggtagtcaacaattcctcacttaacccggccaaactgaaacgccatttggacacaaagcatccgacgcttaaaggcaagagcgaatacttcaaaagaaaatgtaacgagctcaatcaaaagaagcatacttttgagcgatacgtaagggacgataacaagaacctcctgaaagcttcttatctcgtcagtttgagaatagctaaacagggcgaggcatataccatagcggagaagttgatcaagccttgcaccaaggatctgacaacttgcgtatttggagaaaaattcgcgagcaaagttgatctcgtccccctgtccgacacgactatttcaaggcgaatcgaagacatgagttacttctgtgaagccgtgctggtgaacaggttgaaaaatgctaaatgtgggtttacgctgcagatggacgagtcaacagatgttgccggtcttgcaatcctgcttgtgtttgttaggtacatacatgaaagctcttttgaggaggatatgttgttctgcaaagcacttcccactcagacgacaggggaggagattttcaatcttctcaatgcctatttcgaaaagcactccatcccatggaatctgtgttaccacatttgcacagacggtgccaaggcaatggtaggagttattaaaggagtcatagcgagaataaaaaaactcgtccctgatataaaagctagccactgttgcctgcatcgccacgctttggctgtaaagcgaataccgaatgcattgcacgaggtgctcaatgacgctgttaaaatgatcaacttcatcaagtctcggccgttgaatgcgcgcgtcttcgctttgctgtgtgacgatttggggagcctgcataaaaatcttcttcttcataccgaagtgaggtggctgtctagaggaaaggtgctgacccgattttgggaactgagagatgaaattagaattttcttcaacgaaagggaatttgccgggaaattgaacgacaccagttggttgcaaaatttggcatatatagctgacatattcagttatctgaatgaagttaatctttccctgcaagggccgaatagcacaatcttcaaggtaaatagccgcattaacagtattaaatcaaagttgaagttgtgggaagagtgtataacgaaaaataacactgagtgttttgcgaacctcaacgattttttggaaacttcaaacactgcgttggatccaaacctgaagtctaatattttggaacatctcaacggtcttaagaacacctttctggagtattttccacctacgtgtaataatatctcctgggtggagaatcctttcaatgaatgcggtaacgtcgatacactcccaataaaagagagggaacaattgattgacatacggactgatacgacattgaaatcttcattcgtgcctgatggtataggaccattctggatcaaactgatggacgaatttccagaaattagcaaacgagctgtcaaagagctcatgccatttgtaaccacttacctctgtgagaaatcattttccgtctatgtagccacaaaaacaaaatatcgaaatagacttgatgctgaagacgatatgcgactccaacttactactatccatccagacattgacaacctttgtaacaacaagcaggctcagaaatcccactga

SEQ ID NO: 25 - protein, 107 aa, synthetic construct
  REGION 1..107: note = FKBP12
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 26 - DNA, 321 bp, synthetic construct
  misc_feature 1..321: note = FKBP12 - NT
  source 1..321: mol_type = other DNA
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggag

SEQ ID NO: 27 - protein, 5 aa, synthetic construct
  REGION 1..5: note = linker
GGGGS

SEQ ID NO: 28 - DNA, 15 bp, synthetic construct
  misc_feature 1..15: note = linker - NT
  source 1..15: mol_type = other DNA
ggaggaggaggatcc

SEQ ID NO: 29 - protein, 282 aa, synthetic construct
  REGION 1..282: note = truncated caspase 9
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 30 - DNA, 843 bp, synthetic construct
  misc_feature 1..843: note = truncated caspase 9 - NT
  source 1..843: mol_type = other DNA
tttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 31 - protein, 394 aa, synthetic construct
  REGION 1..394: note = inducible proapoptotic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 32 - DNA, 1182 bp, synthetic construct
  misc_feature 1..1182: note = inducible proapoptotic polypeptide - NT
  source 1..1182: mol_type = other DNA
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc